(19) United States
(12) Patent Application Publication
(10) Pub. No.: US 2007/0270954 A1
(43) Pub. Date: Nov. 22, 2007

(54) Human bone substitutional implant

(76) Inventor: Shing-Sheng Wu, Taipei (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/397,695

(22) Filed: Apr. 5, 2006

(51) Int. Cl. A61F 2/44

(52) U.S. Cl. 623/17.11

(57) ABSTRACT

A human bone substitutional implant comprises a main body and a push body. The main body is a hollow cylinder with a thread portion or a ratchet portion disposed on its outer wall. The thread portion or the ratchet portion has at least a slit dividing it into four equal parts. An inner thread is disposed on the inner wall of the thread portion or the ratchet portion. An outer thread is disposed on the outer surface of the push body and corresponds to the inner thread. The push body and the main body are joined together via screw connection of the outer thread and the inner thread. Because the main body has a conical passage therein, the main body will be held open a certain angle to support and enhance a fractured bone of the human body when the push body is axially pushed into the main body.

### BACKGROUND OF THE INVENTION

1. Field of the Invention

The present invention relates to a human bone substitutional implant and, more particularly, to a substitutional implant used for supporting and enhancing a spine, a hip joint or a knee joint of the human body.

2. Description of Related Art

In order to cure spine fracture caused by osteoporosis or external forces, a substitutional implant capable of replacing the fractured spine is usually used. The substitutional implant most commonly used by surgeons in spine surgery is PMMA bone cement, which is the material for replacing the fractured spine at the initial stage.

PMMA bone cement is easily shaped and convenient for use. But its bio-compatibility is bad. It combines with human bone in a non-bone way, and will cause unsettlement of the connection portion at a later stage of use, hence reducing the joint strength. Besides, PMMA bone cement does not solidify and harden by itself so that a solidifying agent is required. A large amount of heat will be produced when PMMA bone cement solidifies and hardens. Moreover, because PMMA bone cement will decompose into poisonous units, it is very harmful to the human body. Therefore, the use of PMMA bone cement in surgery is restricted. Although the problem that bone cement material has a high risk of bio-exclusion has been continually improved, it still has other problems to be solved urgently.

The hip joint is the largest and most important joint of the human body. The weight of the human body is mainly supported by it, and walking actions of the lower limbs also depend on it. Once the hip joint is problematic, one will has the problem in walking or even lose his working capability. The hip joint is the largest joint of the human body. The round head at the upper end of the thigh bone is called the femoral head. Based on statistics, the necrosis rate of the femoral head is unusually high. The necrosis speed of the femoral head may be very slow or very high. Before the femoral head collapses, the primary objective of curing is to save the femoral head by enhancing the blood flow of the femoral head to reduce or avoid the collapse. The applicable methods include high-pressure oxygen, blood fat-reducing drug, anti-coagulant, vasodilatation agent, electric stimulation and decompression surgery. There are also many surgical method used to relieve or avoid the collapse such as bone transplant and bone filling cement.

The above methods, however, have little effects for some people. Once the femoral head has collapsed or even the arthritis comes into being, it is necessary to perform artificial joint surgery. Although artificial joint surgery has a very good effect, reimplant is very likely in the future for an artificial joint implanted in a young person. Or drugs for restraining or destroying bone cells are used to avoid the collapse after the necrosis of the femoral head. Although the initial effect is good, side effects to the human body will easily arise for a long time use of drug.

The knee joint is commonly used in the activity of the human body. Without a healthy knee joint, one's capability of activity will be much limited or he even has a big problem in every step of walking. Therefore, longtime bad posture, bearing a heavy burden or a too high weight will lead to damage of the cartilage of the knee and slight displacement of the joint, hence increasing abrasion and destruction of the cartilage on the surface of the knee joint. Usually, mild-degree pain or degeneration will cure fast after medication or surgery techniques. Although moderate to severe-degree degeneration of the knee joint won't cure, pain of the knee joint can be apparently lessened to improve the activity function of the knee joint and enhance the life quality of the patient. If the knee joint abrades excessively and can't do well for walking and the X-ray photograph shows there is no gap between the joints, it means the cartilage of the joint is severely abraded and destructed. At this time, it is necessary to take artificial knee joint replacement into account. Normal function can be regained only with artificial knee joint replacement. Although the knee joint replacement surgery is more and more popular, there are still some patients whose bones of the knee joint are damaged or have the problem of avascular necrosis so that the artificial knee bone surgery is of no use to them.

Therefore, it is still necessary to improve the above methods of using PMMA bone cement to cure spine fracture caused by osteoporosis or external forces and using the artificial joint replacement surgery or drugs to treat the collapse of the femoral head and knee joint after avascular necrosis.

### SUMMARY OF THE INVENTION

An object of the present invention is to provide a human bone substitutional implant to solve the problems of spine fracture caused by osteoporosis or external forces and bone damage caused by avacular necrosis or trauma of the femoral head and the knee joint. The present invention provides a bone substitutional implant for supporting and enhancing the spine, the femoral head of the hip joint, and the knee joint.

To achieve the above object, the present invention provides a human bone substitutional implant, which comprises a main body and a push body. A thread portion or a ratchet portion is disposed on the outer surface of the main body. One end of the main body has a fixing portion. The thread portion or the ratchet portion has at least a slit. The slit divides the thread portion or the ratchet portion of the main body into four equal parts. An inner thread is disposed on the inner wall of the thread portion or the ratchet portion. An outer thread is disposed on the outer surface of the push body. The outer thread corresponds to the inner thread of the main body. The push body and the main body are joined together by means of screw connection of the outer thread and the inner thread.

### BRIEF DESCRIPTION OF THE DRAWINGS

The various objects and advantages of the present invention will be more readily understood from the following detailed description when read in conjunction with the appended drawing, in which:

FIG. 1 is a plan cross-sectional view of the present invention;

FIG. 2 is a perspective assembly view of the present invention;

FIG. 3A is a first action diagram showing how the present invention is implanted into a vertebral body;

FIG. 3B is a second action diagram showing how the present invention is implanted into a vertebral body of spine;

FIG. 4 is an assembly diagram showing how the present invention is implanted into a vertebral body of spine;

FIG. 5 is a cross-sectional view showing how the present invention is implanted into a femoral head;

FIG. 6 is a perspective assembly view according to a second embodiment of the present invention;

FIG. 7 is a plan cross-sectional view according to a third embodiment of the present invention;

FIG. 8 is a use state diagram according to the third embodiment of the present invention;

FIG. 9 is an exploded perspective view according to a fourth embodiment of the present invention;

FIG. 10 is a plan cross-sectional view according to the fourth embodiment of the present invention; and

FIG. 11 is a plan cross-sectional view according to a fifth embodiment of the present invention.

### DETAILED DESCRIPTION OF THE PREFERRED EMBODIMENTS

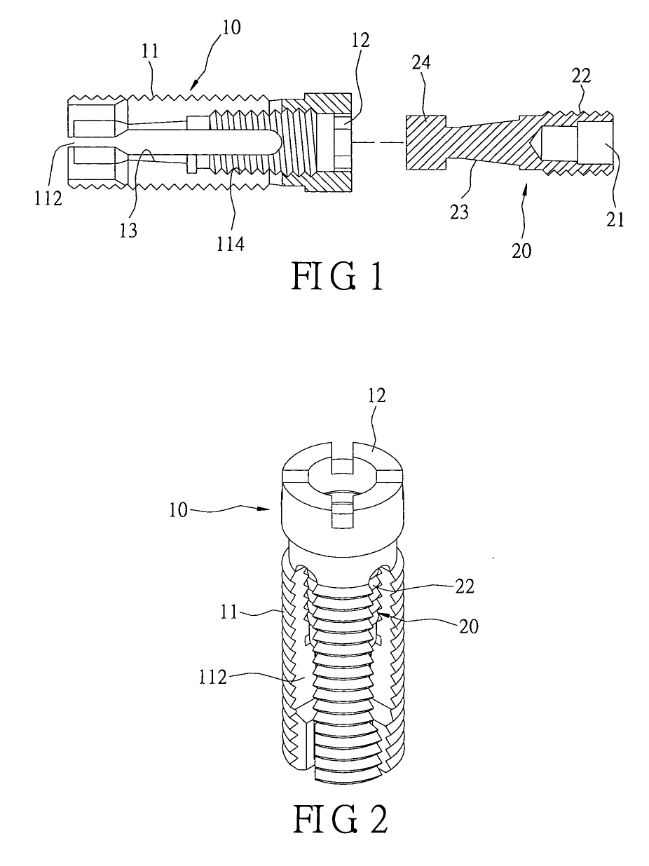

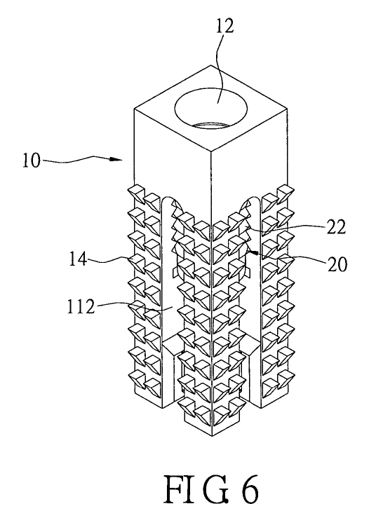

As shown in FIGS. 1 and 2, the present invention provides a human bone substitutional implant, which comprises a main body 10 and a push body 20. The main body 10 is a hollow circular cylinder (or a square cylinder shown in FIG. 6). A conical passage 13 is formed in the main body 10. The diameter of the conical passage 13 gradually diminishes towards the front end of the main body 10. A thread portion 11 (or a ratchet portion 14 shown in FIG. 6) is disposed on an end of the outer wall of the main body 10. The original outer wall is reserved at the other end. A fixing portion 12 is disposed on this other end face and has a groove. The groove is of a straight line shape, a cross shape, a square shape, or a hexagon shape. The thread portion 11 (or the ratchet portion 14) is divided into four equal parts by slits 112. An inner thread 114 is disposed on the inner wall of the main body 10.

One end face of the push body 20 has another groove 21. This groove 21 is of a square shape or a hexagon shape. An outer thread 22 is disposed on the outer wall of the push body 20. The outer thread 22 corresponds to the inner thread 114 of the main body 10. The push body 20 and the main body 10 are joined together by means of screw connection of the outer thread 22 and the inner thread 114. A rod 23 is extended from the front end face of the push body 20. The front end of the rod 23 has a cylinder 24. The cylinder 24 and the rod 23 are integrally formed. The cylinder 24 is pushed axially into the conical passage 13 in the main body 10. Because the diameter of the conical passage 13 diminishes gradually towards the front end of the main body 10 and the thread portion 11 (or the ratchet portion 14) of the main body 10 is divided into four equal parts by the slits 112, the thread portion 11 (or the ratchet portion 14) of the main body 10 will be held open a certain angle when the cylinder 24 of the push body 20 is pushed into the main body 10.

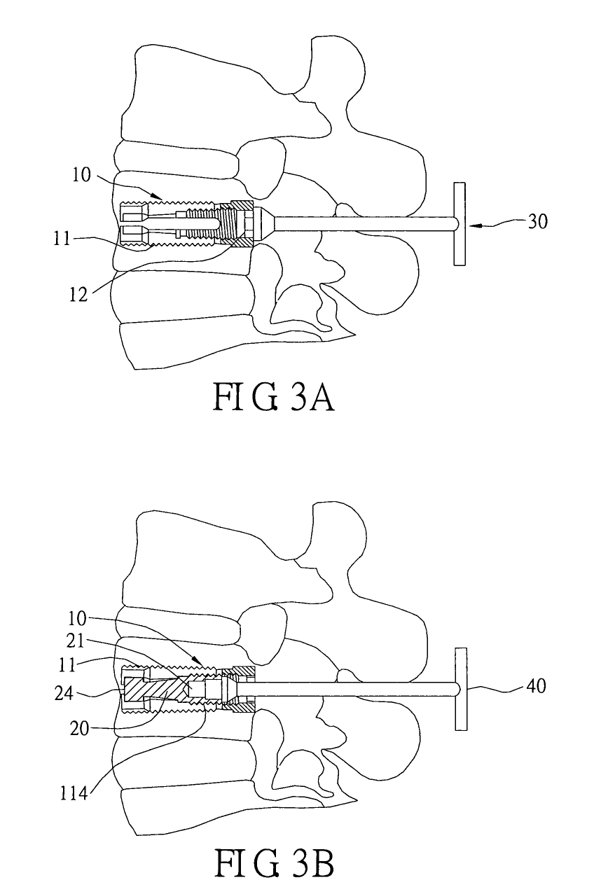

As shown in FIG. 3A, when a surgeon implants the present invention into a vertebral body of spine, he first uses a first hand tool 30 matched with the fixing portion 12 of the main body 10 to screw (or push) the main body 10 into the vertebral body of the spine through the screw portion 11 (or the ratchet portion 14) of the main body 10. He then uses a second hand tool 40 matched with the groove 21 of the push body 20 to turn the push body 20 for screwing the push body 20 and the inner thread 114 of the main body 10 together. The cylinder 24 at the front end of the push body 20 is thus pushed axially into the main body 10 to hold open the thread portion 11 (or the ratchet portion 14) of the main body 10 a certain angle, thereby supporting and enhancing the vertebral body of the spine.

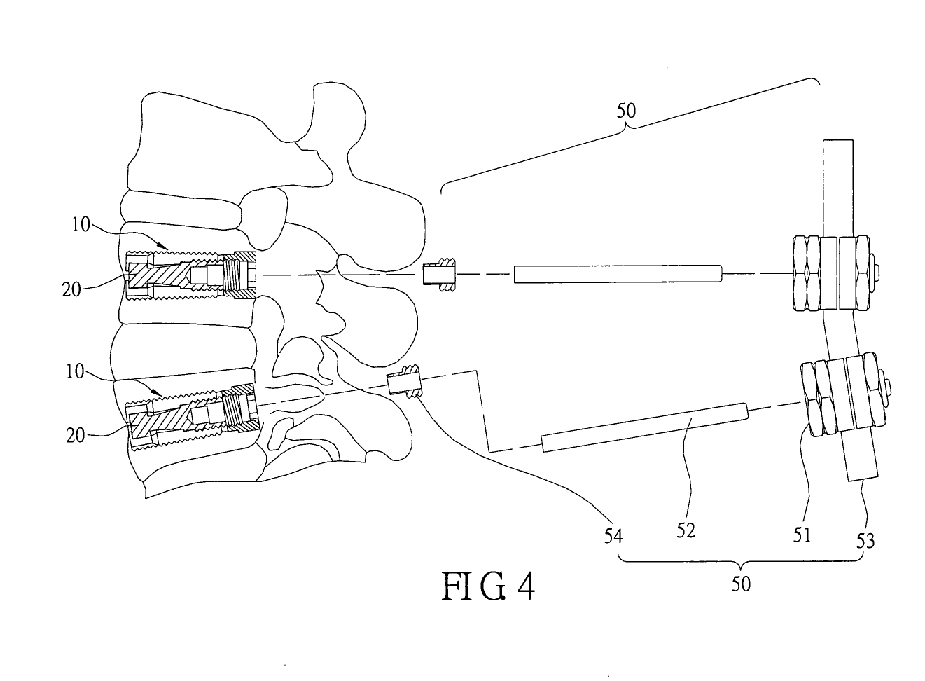

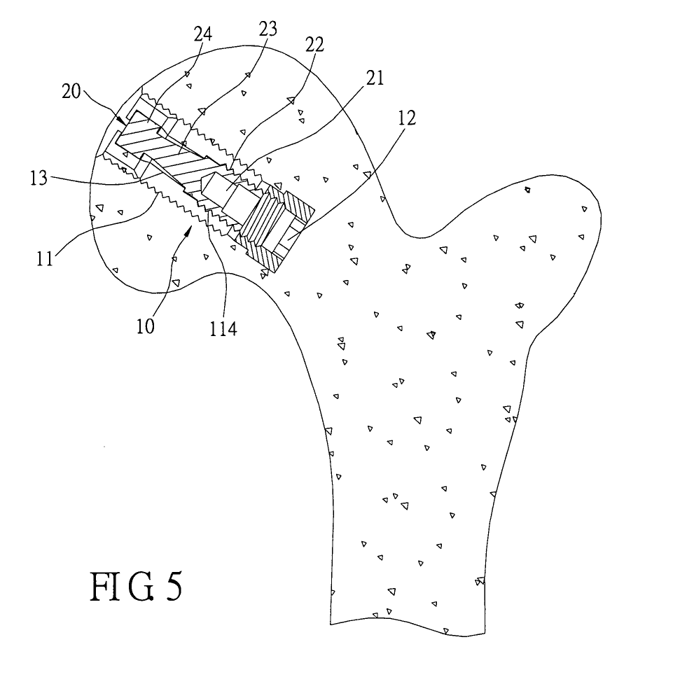

As shown in FIG. 4, a spine of the human body is composed of a plurality of vertebrae. Spine fracture caused by osteoporosis often occurs at different vertebrae. After the human bone substitutional implants of the present invention are implanted into several fractured vertebrae, connection subassemblies 50 can be used to connect these human bone substitutional implants together for supporting and enhancing these fractured vertebrae. The connection subassembly 50 comprises a connection portion 51 for fixing a connection rod 52 and a transverse rod 53. The connection rod 52 is fixed in the main body 10 by using a locking component 54. The transverse rod 53 is matched with another connection subassembly 50 to connect another human bone substitutional implant of the present invention fixed on another vertebra together, thereby supporting and enhancing several fractured vertebrae.

As shown in FIG. 5, before the present invention is implanted into a femoral head in a spiral way, it is necessary to first drill a hole in the collapse femoral head to reduce the internal stress of the bone structure. The present invention is then implanted into the avascular necrosis of femoral head in the way stated above to provide a supporting force for the avascular necrosis of femoral head, thereby avoiding continual collapse of the femoral head.

As shown in FIG. 6, the main body 10 is a hollow square cylinder. The inside of the main body 10 is the same as that of the main body of a circular cylinder shape (as shown in FIG. 1). A ratchet portion 14 is disposed on an end of the outer wall of the main body 10. The original outer wall is reserved at the other end. A fixing portion 12 is disposed on this other end face. The fixing portion 12 is circular. The ratchet portion 14 is divided into four equal parts by slits 112. When the push body 20 is pushed into the main body 10, the ratchet portion 14 will be held open a certain angle.

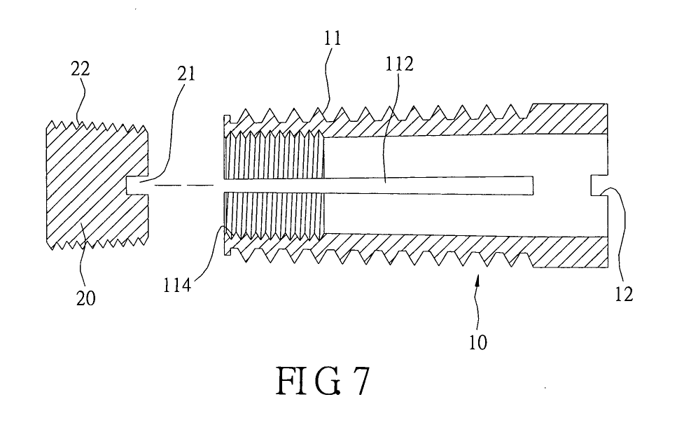

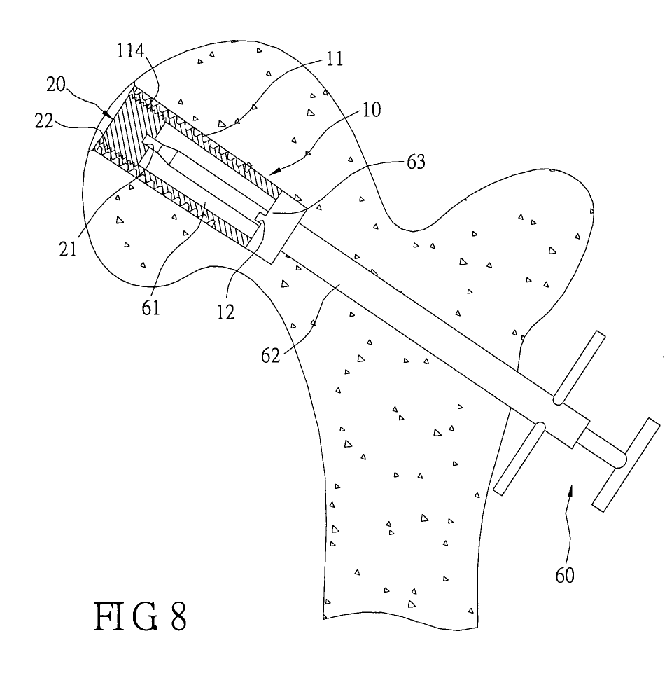

As shown in FIGS. 7 and 8, the push body 20 is designed to be a cone having a certain cone angle. When a surgeon wants to implant the present invention into a spine, a femoral head, or femoral condylar of a knee joint of the human body, he will use a third hand tool 60 composed of an inner rod 61 and an outer rod 62. A bump 63 matched with the fixing portion 12 is disposed at the distal end of the outer rod 62. The bump 63 makes the main body 10 immobile. The outer rod 62 is used to implant the present invention into the bones of the human body in a spiral way. Through the distal end of the inner rod 61 matched with the groove 21 of the push body 20, the push body 20 is turned to screw the outer thread 22 of the push body 20 and the inner thread 114 of the main body 10 together. Because the thread portion 11 of the main body 10 is divided into four equal parts by the slits 112 and the push body 20 has an appropriate cone angle, the thread portion 11 of the main body 10 will be held open a certain angle when the push body 20 and the inner thread 114 are screwed together. After the push body 20 is completely screwed into the main body 10, the open angle of the main body 10 is the cone angle of the push body 20. Moreover, the front end face of the main body 10 aligns with the front end face of the push body 20 so that the bump 63 of the outer rod 62 can be separated from the fixing portion 12 of the main body 10. The installation of the present invention is thus finished to provide a supporting force for and avoid continual harm to the fractured bone of the human body.

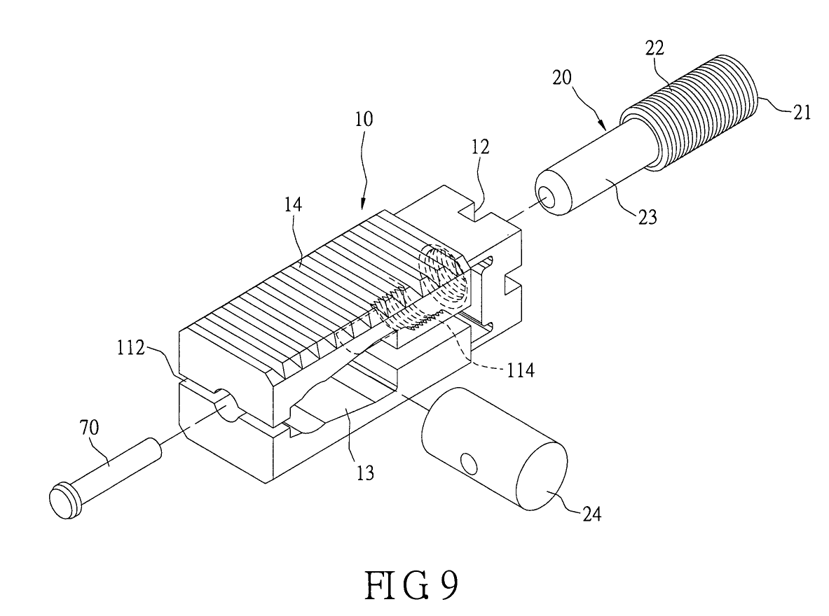

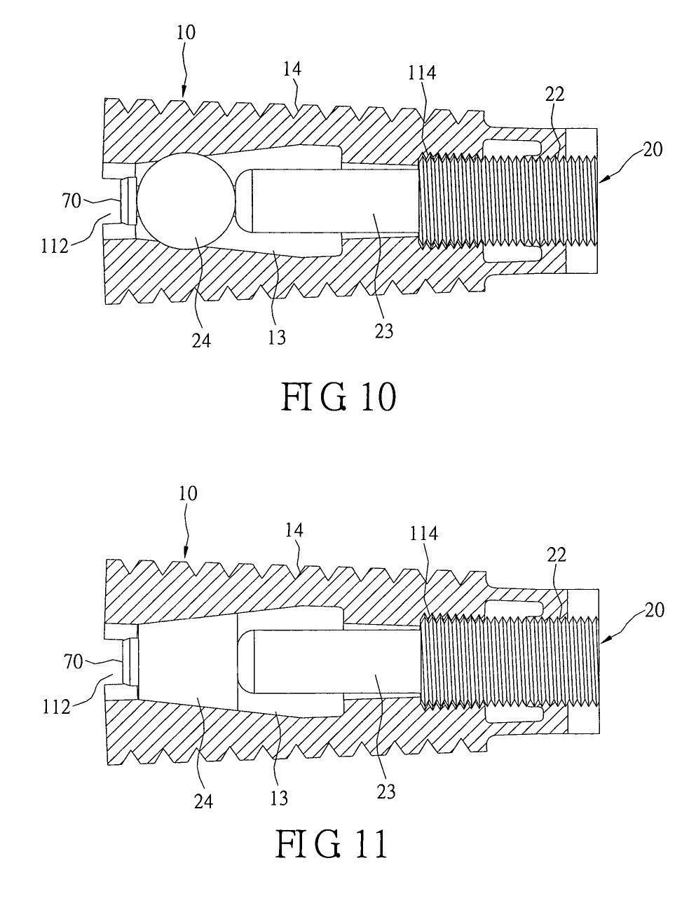

As shown in FIGS. 9 and 10, the main body 10 is a hollow square cylinder. The slit 112 divides the main body 10 into two equal parts. A ratchet portion 14 is disposed on the outer surface of the main body 10. The cylinder 24 is a circular. A fixing component 70 is used to connect the cylinder 24 and the rod 23 together. The circular cylinder 24 is used to hold open the main body 10.

As shown in FIG. 11, the cylinder 24 is square. Every two opposite outer surfaces of the cylinder 24 have a certain slanting angle. This slanting angle is designed to help the square cylinder 24 in the conical passage 13 hold open the main body 10.

To sum up, the present invention has the following characteristics:
1. The present invention is a human bone substitutional implant. A thread portion 11 is disposed on the outer wall of a main body 10. Based on the principle of thread, the present invention can be implanted into a spine, a femoral head, and the femoral condylar of a knee joint of the human body. That is, the present invention has a convenient implanting manner, and can enhance the efficiency of surgery. Moreover, through the limit function of thread in the reverse direction, the present invention can be prevented from going out of the fractured bone by external forces.
2. The present invention is a human bone substitutional implant. A ratchet portion 14 is disposed on the outer wall of a main body 10. Using the ratchet portion 14, the present invention can be implanted into a spine, a femoral head, and the femoral condylar of a knee joint of the human body. That is, the present invention has a convenient implanting manner, and can enhance the efficiency of surgery. Moreover, through the limit function of ratchet in the reverse direction, the present invention can be prevented from going out of the fractured bone by external forces.
3. The push body 20 and the main body 10 of the present invention are matched to hold open the main body 10 a certain angle for providing a good supporting force for a patient's spine to avoid continual collapse of the spine or providing supporting and reinforcing effects for a femoral head or femoral condylar of a knee joint.

Although the present invention has been described with reference to the preferred embodiment thereof, it will be understood that the invention is not limited to the details thereof. Various substitutions and modifications have been suggested in the foregoing description, and other will occur to those of ordinary skill in the art. Therefore, all such substitutions and modifications are intended to be embraced within the scope of the invention as defined in the appended claims.

What is claimed is:

1. A human bone substitutional implant comprising:
a main body having at least a slit, an inner thread being disposed on an inner wall of said main body; and
a push body with an outer thread formed on an outer surface thereof, said outer thread corresponding to said inner thread of said main body, said push body and said main body being joined together by means of screw connection of said outer thread and said inner thread.

2. The human bone substitutional implant as claimed in claim 1, wherein said main body is a hollow circular cylinder, a thread portion is disposed on an outer surface of said main body, and said slit divides said thread portion into four equal parts.

3. The human bone substitutional implant as claimed in claim 1, wherein said main body is a hollow square cylinder, a ratchet portion is disposed on an outer surface of said main body, and said slit divides said ratchet portion into four equal parts.

4. The human bone substitutional implant as claimed in claim 1, wherein said main body is a hollow cuboid, said slit divides said main body into two equal parts, and a ratchet portion is disposed on an outer surface of said main body.

5. The human bone substitutional implant as claimed in claim 1, wherein a fixing portion is formed on an end face of said main body, and said fixing portion is of a straight line shape, a cross shape, a square shape, a hexagon shape, or a circular shape.

6. The human bone substitutional implant as claimed in claim 5, wherein a groove is disposed on an end face of said push body, and said groove is of a square shape or a hexagon shape.

7. The human bone substitutional implant as claimed in claim 6, wherein a rod is extended from an end of said push body, one end of said rod has a cylinder, a conical passage is formed in said main body, and said main body is held open a certain angle when said cylinder is axially pushed into said conical passage.

8. The human bone substitutional implant as claimed in claim 7, wherein said cylinder is a circular or square cylinder, and said cylinder and said rod are integrally formed or a fixing component is used to join said cylinder and said rod together.

9. The human bone substitutional implant as claimed in claim 8, further comprising a connection subassembly, wherein said subassembly comprising a connection portion for fixing a connection rod and a transverse rod, said connection rod being fixed in said main body through a locking component.

10. The human bone substitutional implant as claimed in claim 6, wherein said push body is a cone having a certain cone angle.
